(19) United States
(12) Patent Application Publication
(10) Pub. No.: US 2022/0101458 A1
(43) Pub. Date: Mar. 31, 2022

(54) INFORMATION PROCESSING DEVICE, INFORMATION PROCESSING SYSTEM, INFORMATION PROCESSING METHOD, AND PROGRAM

(71) Applicant: TOYOTA JIDOSHA KABUSHIKI KAISHA, Toyota-shi (JP)

(72) Inventors: Toyokazu NAKASHIMA, Nagoya-shi (JP); Takayuki HATORI, Nagakute-shi (JP); Shuichi SAWADA, Nagoya-shi (JP); Daiki KUBO, Toyota-shi (JP); Tomoki ISHIKAWA, Nagoya-shi (JP); Naoya OKA, Nagakute-shi (JP)

(73) Assignee: TOYOTA JIDOSHA KABUSHIKI KAISHA, Toyota-shi (JP)

(21) Appl. No.: 17/470,083

(22) Filed: Sep. 9, 2021

(30) Foreign Application Priority Data
Sep. 30, 2020 (JP) 2020-166259

(51) Int. Cl. G06Q 50/12; G01N 33/497

(52) U.S. Cl. CPC G06Q 50/12; G01N 33/4972

(57) ABSTRACT

An information processing device including a controller configured to execute detecting an alcohol contained in exhaled breath of a user who has returned home by using an alcohol sensor, and estimating consumption of an alcoholic beverage by the user after returning home based on a result of the detection of the alcohol.

### CROSS-REFERENCE TO RELATED APPLICATION

This application claims priority to Japanese Patent Application No. 2020-166259 filed on Sep. 30, 2020, incorporated herein by reference in its entirety.

### BACKGROUND

### 1. Technical Field

The disclosure relates to a technique of assisting a user.

### 2. Description of Related Art

There is technology of assisting the regular purchase of goods, such as daily commodities. For example, Japanese Unexamined Patent Application Publication No. 2016-224650 (JP 2016-224650 A) discloses a system that executes processing of ordering a product related to a beacon device, triggered by an order button provided on the beacon device being pressed.

By installing the beacon device described in Japanese Unexamined Patent Application Publication No. 2016-224650 (JP 2016-224650 A) in the vicinity of a location in which the product is stored, such as a refrigerator or a washroom, placing an order for the product by a simple operation is possible.

### SUMMARY

On the other hand, in the related art, there is room for improvement in that the user has to manage the stock of the product and place an order for the product by himself or herself. In order to automate stock management, to know how much a target product has been consumed by the user is needed.

The disclosure is to estimate the consumption of an alcoholic beverage by the user.

A first aspect of the present disclosure relates to an information processing device including a controller. The controller is configured to execute detecting an alcohol contained in exhaled breath of a user who has returned home by using an alcohol sensor, and estimating consumption of an alcoholic beverage by the user after returning home based on a result of the detection of the alcohol.

A second aspect of the present disclosure relates to an information processing system including an alcohol sensor, and a controller. The alcohol sensor is installed at home of a user and configured to detect an alcohol contained in exhaled breath of the user. Specifically, the controller is configured to execute detecting the alcohol contained in the exhaled breath of the user who has returned home by using the alcohol sensor, and estimating consumption of an alcoholic beverage by the user after returning home based on a result of the detection of the alcohol.

A third aspect of the present disclosure relates to an information processing method including a step of detecting an alcohol contained in exhaled breath of the user who has returned home, and a step of estimating consumption of an alcoholic beverage by the user after returning home based on a result of the detection of the alcohol.

Another aspect of the present disclosure relates to a program that causes a computer to execute the information processing method, or a computer-readable storage medium that non-transitory stores the program.

According to the disclosure, the consumption of the alcoholic beverage by the user can be estimated.

### BRIEF DESCRIPTION OF THE DRAWINGS

Features, advantages, and technical and industrial significance of exemplary embodiments of the disclosure will be described below with reference to the accompanying drawings, in which like signs denote like elements, and wherein:

FIG. 1 is a diagram showing an outline of an information processing system;

FIG. 2 is a diagram showing in detail components of the information processing system according to a first embodiment;

FIG. 3 is a view showing a disposed position of a sensor within doors;

FIG. 4 is a table showing concentration data stored in a storage unit;

FIG. 5A is a graph showing a concentration of an alcohol in air;

FIG. 5B is a graph showing the concentration of the alcohol in the air;

FIG. 6A is a table showing estimation data in the first embodiment;

FIG. 6B is a table showing the estimation data in the first embodiment;

FIG. 7A is a table showing stock data stored in the storage unit;

FIG. 7B is a table showing the stock data stored in the storage unit;

FIG. 7C is a table showing the stock data stored in the storage unit;

FIG. 8 is a diagram showing a data flow between modules included in a controller;

FIG. 9 is a flowchart of processing executed by the controller in the first embodiment;

FIG. 10A is a table showing estimation data in a second embodiment; and

FIG. 10B is a graph showing the estimation data in the second embodiment.

### DETAILED DESCRIPTION OF EMBODIMENTS

There is known a system of applying for the purchase of a predetermined product, such as daily commodities and an alcoholic beverage, based on predetermined operation by a user. However, in such a system, the user has to manage the stock of the product and place an order for the product.

In order to solve this problem, an information processing device according to the present embodiment estimates the consumption of the alcoholic beverage by the user based on the result of sensing the user.

Specifically, the information processing device includes the controller is configured to execute detecting the alcohol contained in the exhaled breath of the user who has returned home by using the alcohol sensor, and estimating consumption of an alcoholic beverage by the user after returning home based on a result of the detection of the alcohol.

The presence or absence of the alcohol contained in the exhaled breath of the user can be determined, for example, by a sensor (alcohol sensor) that detects the alcohol in the air. It is preferable that the alcohol sensor be installed at a position in which the alcohol contained in the exhaled breath of the user immediately after the user has returned home can be detected, for example.

The controller estimates the consumption of the alcoholic beverage by the user after returning home based on the alcohol contained in the exhaled breath of the user who has returned home. For example, in a case where a sufficient amount of the alcohol is detected in the exhaled breath of the user when the user has returned home, the estimation is made that the user does not drink any more alcohol during the day. On the other hand, in a case where the user who has a habit of drinking the alcohol after returning home has not consumed the alcohol until the user returns home, the estimation is made that the user drinks the alcohol at home. In this way, an amount of alcohol drinking (consumption of alcoholic beverage) by the user after returning home can be estimated based on the presence or absence of the alcohol in the exhaled breath when the user has returned home.

Further, the controller may be configured to perform the detection by using the alcohol sensor that is installed at an entrance of home of the user.

By using the sensor installed at the entrance, the exhaled breath of the user who has returned home can be captured.

Further, the controller may be configured to, in a case where the alcohol is not detected from the exhaled breath of the user, estimate that a predetermined amount of the alcoholic beverage is consumed by the user after returning home.

Further, the controller may be configured to estimate that an amount of the alcoholic beverage is consumed by the user after returning home is different between a case where the alcohol is detected from the exhaled breath of the user and a case where the alcohol is not detected from the exhaled breath of the user.

The estimated consumption of the alcoholic beverage can be decided in advance based on the habit of drinking alcohol of the user.

Further, the controller may be configured to acquire a concentration of the alcohol contained in the exhaled breath of the user by using the alcohol sensor.

The concentration of the alcohol in the exhaled breath may be directly or indirectly acquired. For example, in a case where the exhaled breath of the user does not directly contact the sensor, the concentration of the alcohol contained in the exhaled breath may be calculated based on the concentration of the alcohol in the air around the sensor. By using the concentration of the alcohol in the exhaled breath, the estimation can be made how much alcohol the user drinks after returning home.

Further, the controller may be configured to, in a case where the alcohol is detected from the exhaled breath of the user, estimate that an amount of the alcoholic beverage corresponding to the acquired concentration of the alcohol is consumed by the user after returning home.

For example, the estimation can be made that the amount of alcohol drinking at home is larger as the concentration of the alcohol in the exhaled breath is lower when the user has returned home.

Further, the information processing device may further include a storage unit configured to store a model for estimating the consumption of the alcoholic beverage by the user after returning home based on the acquired concentration of the alcohol.

By using a model in which the acquired concentration of the alcohol is associated with the amount of alcohol drinking by the user after returning home, the accuracy of the estimation can be improved. It is preferable that the model be generated or updated based on the habit of drinking alcohol of the user.

Further, the controller may be configured to further acquire an actual value of the consumption of the alcoholic beverage consumed by the user after returning home.

Further, the model may be updated based on the concentration of the alcohol acquired when the user has returned home and the actual value.

By feeding back the actual value of the amount of alcohol drinking by the user, more accurate estimation is possible. The actual value may be input by the user himself or herself, or may be generated by the device based on the result of sensing the user.

Further, the controller may be configured to perform the detection in a predetermined period of time when the user is expected to return home, and perform the estimation based on a result of the detection in the predetermined period of time.

By acquiring the data at the period of time when the user is expected to return home, the estimation can be made without sensing that the user has returned home.

Embodiments of the present disclosure will be described below based on the drawings. The configuration of the following embodiments is an exemplification, and the present disclosure is not limited to the configuration of the embodiments.

### First Embodiment

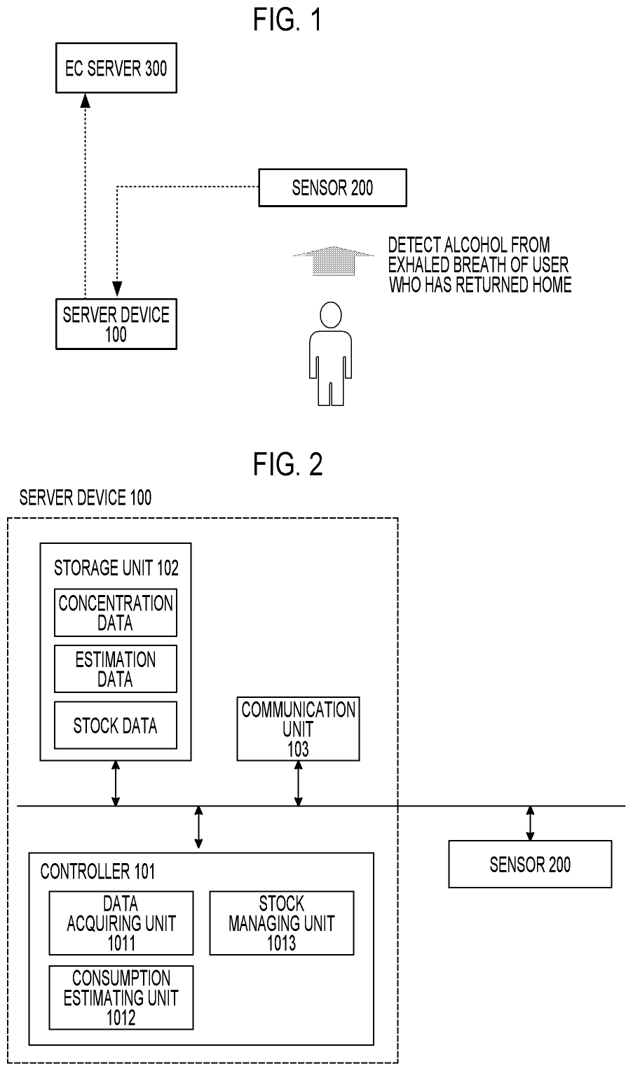

An outline of the information processing system according to the first embodiment will be described with reference to FIG. 1. The information processing system according to the present embodiment is configured by including a server device 100 that is installed at home of the user, a sensor 200 that senses the user, and an EC server 300 that accepts an order for the alcoholic beverage.

The sensor 200 is a sensor that detects the concentration of the alcohol in the air. The sensor 200 is installed at the entrance of home of the user and can detect the alcohol contained in the exhaled breath of the user who has returned home.

The server device 100 executes processing of estimating whether or not the user consumes (that is, drinks) the alcoholic beverage after returning home and estimating the consumption based on data acquired via the sensor 200. In addition, the server device 100 executes processing of updating the stock amount of the alcoholic beverage at home and processing of placing an order for the alcoholic beverage as needed based on the estimation result.

The EC server 300 is a device that accepts an order for the product (alcoholic beverage) via a network. The EC server 300 is managed by a business operator who operates a mail-order business.

The server device 100 may be installed in home of the user, but may also be installed in a remote location. Also, one server device 100 may control a plurality of users.

FIG. 2 is a diagram showing in more detail components of the information processing system according to the present embodiment. Here, the sensor 200 will be described first.

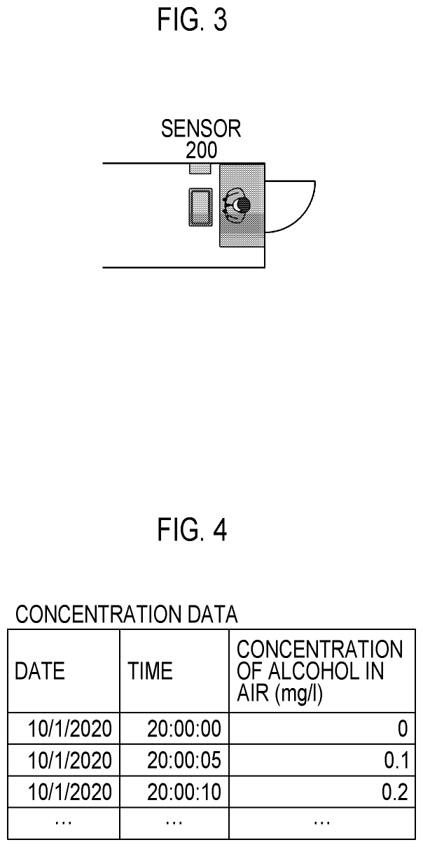

The sensor 200 is an alcohol sensor that detects the concentration of the alcohol in the air. The sensor 200 may be of any kind and installation location as long as the sensor can detect the alcohol contained in the exhaled breath of the user. FIG. 3 is a plan view showing home entrance of the user. As shown in FIG. 3, the sensor 200 can be installed in the vicinity of the entrance.

In the present embodiment, the sensor 200 is installed in the vicinity of the entrance, but the sensor 200 may be installed in another location. For example, the sensor 200 may be installed outside the entrance door, or may be provided inside a vehicle in a case where the user gets on an autonomous traveling vehicle and returns home.

The server device 100 estimates the consumption of the alcoholic beverage by the user after returning home based on the data acquired from the sensor 200 (hereinafter, sensor data). Further, the server device 100 manages the stock amount of the alcoholic beverage at home of the user and places an order for the alcoholic beverage as needed.

The server device 100 can be configured by a general purpose computer. That is, the server device 100 can be configured as a computer including a processor such as a CPU or a GPU, a main storage device such as a RAM or a ROM, and an auxiliary storage device such as an EPROM, a hard disk drive, or a removable medium. The removable medium may be, for example, a USB memory or a disk recording medium such as a CD or a DVD. An operating system (OS), various programs, various tables, and the like are stored in the auxiliary storage device, the program stored in the auxiliary storage device is loaded into a work area of the main storage device, the programs are execute, and the configuration units are controlled through the execution of the program, so that each function that meets a predetermined purpose as described below can be realized. Note that, a part or all of the functions may be realized by a hardware circuit such as an ASIC and a FPGA.

A controller 101 is an arithmetic device that controls the control performed by the server device 100. The controller 101 can be realized by an arithmetic processing device such as the CPU.

The controller 101 is configured by including three functional modules of a data acquiring unit 1011, a consumption estimating unit 1012, and a stock managing unit 1013. Each of the functional modules may be realized by executing the stored program by the CPU.

The data acquiring unit 1011 acquires the sensor data from the sensor 200. The acquired sensor data indicates the concentration of the alcohol in the air around the sensor 200 (that is, around the user immediately after returning home). The sensor data acquired by the data acquiring unit 1011 is sequentially accumulated as concentration data in a storage unit 102 described below. The concentration data is data indicating the transition of the concentration of the alcohol in the air.

The consumption estimating unit 1012 estimates the consumption of the alcoholic beverage by the user after returning home based on the stored concentration data.

Specifically, the consumption estimating unit 1012 determines whether or not the user has drunk the alcohol while going out (before returning home), and estimates the amount of alcohol drinking after returning home (consumption of alcoholic beverage) based on the result of the determination and estimation data described below. The estimated consumption is transmitted to the stock managing unit 1013.

The stock managing unit 1013 manages data (stock data) indicating the stock amount of the alcoholic beverage at home of the user, and updates the stock data based on the consumption of the alcoholic beverage estimated by the consumption estimating unit 1012. In addition, the stock managing unit 1013 places an order for the alcoholic beverage to the EC server 300 in a case where the stock amount of the alcoholic beverage is less than a predetermined value.

The storage unit 102 is configured by including the main storage device and the auxiliary storage device. The main storage device is a memory in which a program executed by the controller 101 and data used in the control program are expanded. The auxiliary storage device is a device in which the program executed by the controller 101 and the data used in the control program are stored.

The storage unit 102 stores the concentration data described above. FIG. 4 shows an example of the concentration data. The concentration data includes the date and time when the sensor 200 acquires the sensor data, and the concentration of the alcohol in the air obtained by sensing.

In addition, the storage unit 102 stores data (estimation data) for estimating the consumption of the alcoholic beverage by the user after returning home. The estimation data in the first embodiment is data in which the presence or absence of the alcohol detection is associated with the amount of the alcoholic beverage that is expected to be consumed by the user after returning home. In the present specification, the consumption of the alcoholic beverage by the user is estimated for the period from when the user returns home to when the user goes to bed.

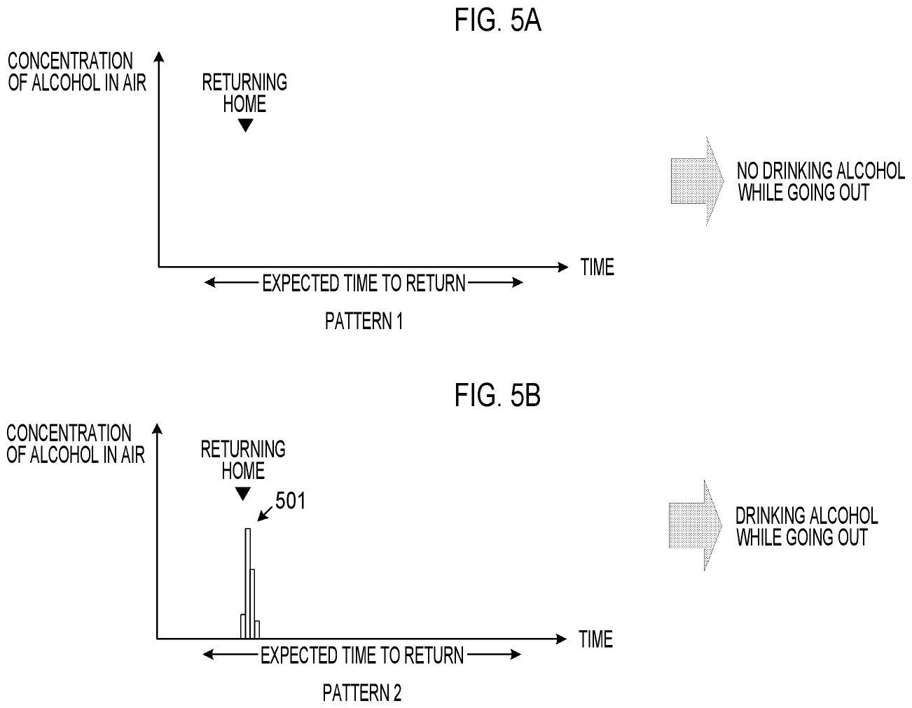

Here, a method of estimating the consumption of the alcoholic beverage after returning home will be described with reference to FIG. 5A and FIG. 5B. FIG. 5A and FIG. 5B are graphs showing the change with time of the concentration of the alcohol in the air detected by the sensor 200.

For example, in a case where the user does not drink the alcohol while going out, the sensor 200 does not detect the alcohol before and after returning home. That is, the change with time of the concentration of the alcohol in the air is as shown in FIG. 5A.

On the other hand, in a case where the user drinks the alcohol while going out, the sensor 200 detects the alcohol before and after returning home. In this case, the change with time of the concentration of the alcohol in the air is as shown in FIG. 5B.

In a case where the sensor 200 detects the alcohol in the period of time when the user is expected to return home, the controller 101 can determine that the user has drunk the alcohol before returning home.

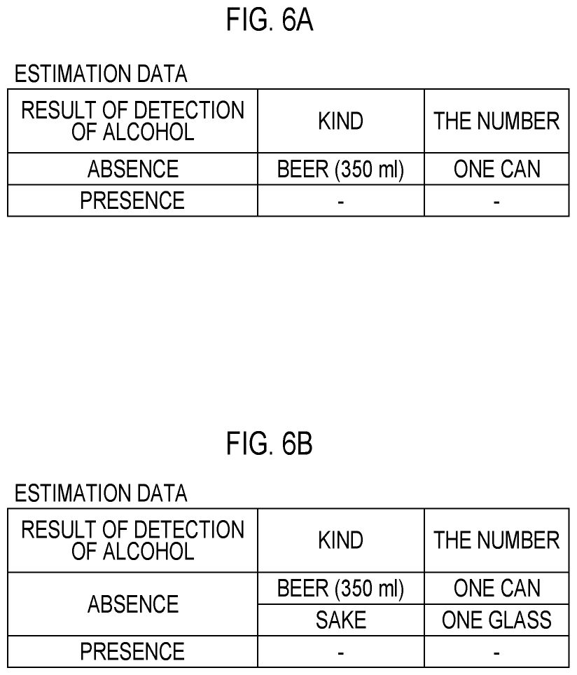

FIG. 6A is a table showing the estimation data. This example shows that the user consumes one can of beer (350 ml) after returning home in a case where the alcohol is not detected when the user has returned home. In addition, this example shows that the user does not drink the alcohol after returning home in a case where the alcohol is detected when the user has returned home. In this example, one kind of the alcoholic beverage is shown, but as shown in FIG. 6B, the user may consume a plurality of kinds of the alcoholic beverages.

The estimation data corresponding to the user is generated in advance and stored in the storage unit 102. The estimation data may be in a table format as shown, or may be data in another format. In addition, the estimation data may be generated based on the self-report by the user, or may be generated based on the result of sensing the user.

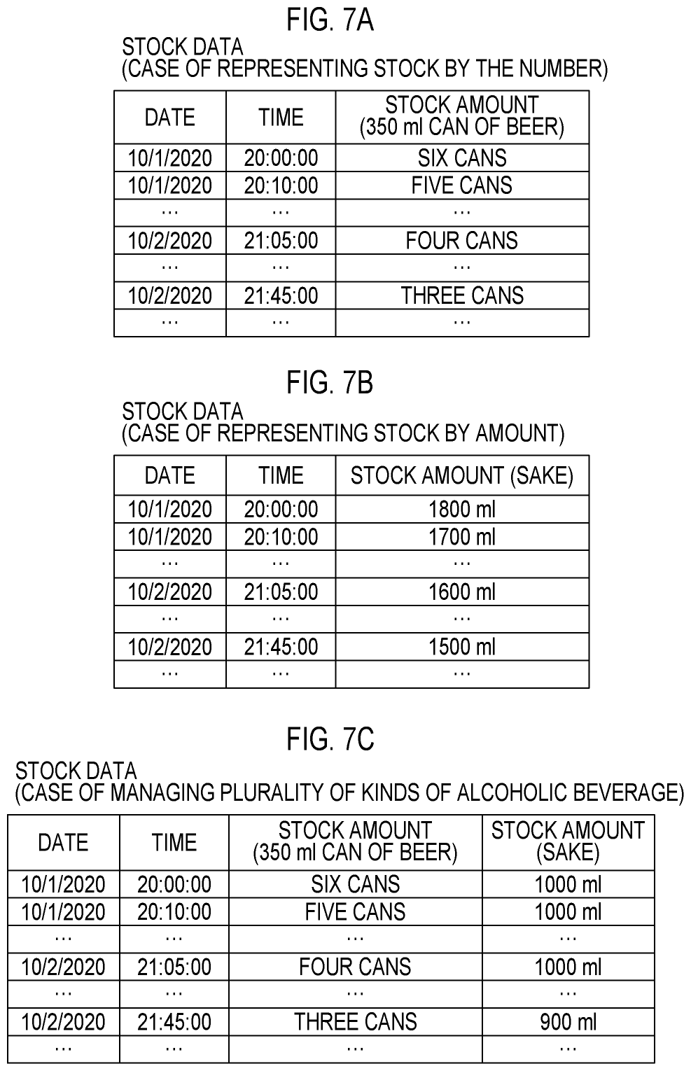

In addition, the storage unit 102 stores data (stock data) for managing the stock of the alcoholic beverage at home of the user. FIG. 7A to FIG. 7C show examples of the stock data. The stock data is data in which the stock amount of the alcoholic beverage under management is associated with the date and time.

The stock amount of the alcoholic beverage may be represented for each kind. For example, in a case where there are the kinds of the alcoholic beverages consumed by the user, the stock amount for each kind may be held. FIG. 7A and FIG. 7B show the examples of the stock data in a case where there is one kind of the alcoholic beverage. Further, FIG. 7C shows the example of the stock data in a case where there are the kinds of the alcoholic beverages. The stock data is updated in a case where the alcoholic beverage are consumed and a case where the alcoholic beverage is purchased.

A communication unit 103 is a communication interface that connects the server device 100 to the network. The communication unit 103 is configured by including, for example, a network interface board and a wireless communication circuit for wireless communication.

The configuration shown in FIG. 2 is an example, and all or a part of the shown functions may be executed by using a circuit exclusively designed. Further, the program may be stored or executed by a combination of the main storage device and the auxiliary storage device other than those shown in FIG. 2.

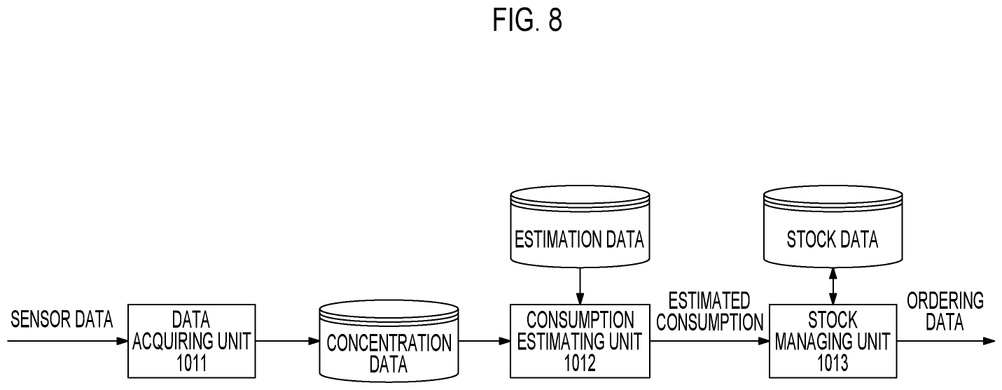

Next, the processing performed by the controller 101 will be described with reference to FIG. 8 that is a diagram showing the data transmitted and received between the modules.

The data acquiring unit 1011 receives the sensor data from the sensor 200 installed at the entrance, and accumulates the sensor data as the concentration data in the storage unit 102. As described above, the concentration data is data showing the change with time of the concentration of the alcohol in the air.

The concentration of the alcohol in the air detected by the sensor 200 may be recorded as it is in the concentration data, or may be recorded after correcting an acquired value. For example, in a case where the sensor 200 does not directly sense the exhaled breath of the user, the concentration of the alcohol in the exhaled breath of the user may be calculated based on the concentration of the alcohol in the air around the sensor, and the calculated value may be stored.

The consumption estimating unit 1012 estimates the consumption of the alcoholic beverage by the user who has returned home based on the acquired concentration data. Specifically, the consumption estimating unit 1012 determines whether the pattern “alcohol is detected” or the pattern “alcohol is not detected” is applicable, and acquires the consumed amount of the alcoholic beverage associated with the pattern.

For example, in the example of FIG. 6A, in a case where the sensor 200 does not detect the alcohol, the estimation can be made that the user drinks one can of beer (350 ml) after returning home. Further, in the example of FIG. 6A, in a case where the sensor 200 detects the alcohol, the estimation can be made that the user does not drink the alcohol after returning home.

Further, in the example of FIG. 6B, in a case where the sensor 200 does not detect the alcohol, the estimation can be made that the user drinks one can of beer (350 ml) and one glass of sake after returning home.

The determination result is transmitted to the stock managing unit 1013. In a case where there is one kind of the alcoholic beverage managed as stock, the consumption of the alcoholic beverage (for example, “350 ml can×one”) is transmitted as the determination result. In a case where there are the kinds of the alcoholic beverages managed as stock, the kind and consumption (for example, “can of beer (350 ml can)×one” is transmitted.

The stock managing unit 1013 updates the stock data based on information transmitted from the consumption estimating unit 1012. Specifically, the consumption is subtracted from the current stock amount, and a new record is added. In a case where there are the kinds of the alcoholic beverages managed as stock, the stock amount of the corresponding kind is subtracted.

Further, the stock managing unit 1013 executes processing of placing an order for the alcoholic beverage in a case where the stock amount of the alcoholic beverage is less than a predetermined standard. For example, data (ordering data) for placing an order for a predetermined kind and a predetermined amount of the alcoholic beverage is generated and transmitted to a predetermined EC server 300.

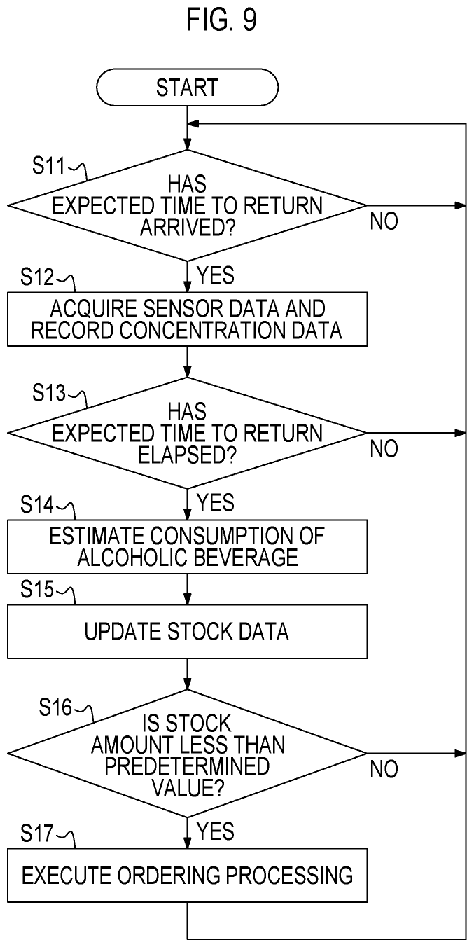

FIG. 9 is a flowchart of processing performed by the server device 100. The shown processing is periodically executed while the server device 100 is running.

First, in step S11, the data acquiring unit 1011 determines whether or not the expected time to return home corresponding to the user has arrived. The expected time to return home corresponding to the user is set in advance. For example, in a case where the fact that the user returns home between 18:00 and 21:00 every day is set, positive determination is made after 18:00. In a case where the expected time to return home has not arrived, the same processing is repeated after waiting for a predetermined time.

Next, in step S12, the data acquiring unit 1011 acquires the sensor data from the sensor 200, generates and adds a new record of the concentration data based on the acquired sensor data.

Next, in step S13, the determination is made as to whether or not the expected time to return home has elapsed. In the case of the example described above, positive determination is made after 21:00. In a case where the expected time to return home has not elapsed, the processing returns to step S11, and the acquisition of the sensor data is repeated at a predetermined cycle. In a case where the expected time to return home has elapsed, the processing proceeds to step S14.

In step S14, the consumption estimating unit 1012 estimates the consumption of the alcoholic beverage by the user who has returned home. In this step, the concentration data and the estimation data stored in the storage unit 102 are used to decide the kind and the amount of the alcoholic beverage estimated to be consumed by the method described above. That is, in a case (1) where the alcohol is detected from the exhaled breath of the user who has returned home, the estimation is made that the user does not drink the alcohol after returning home, and in a case (2) where the alcohol is not detected from the exhaled breath of the user who has returned home, the estimation is made that the user drinks a predetermined amount of the alcohol after returning home.

The determination result is transmitted to the stock managing unit 1013.

In step S15, the stock managing unit 1013 updates the stock data based on a result of the estimation performed by the consumption estimating unit 1012.

In step S16, the stock managing unit 1013 determines whether or not any of the alcoholic beverages under management has a stock amount less than the predetermined value. Here, in a case where the stock amount is less than the predetermined value, the data (ordering data) for placing an order for the corresponding beverage is generated and transmitted to the EC server 300 (step S17). In a case where the stock amount is not less than the predetermined value, the processing returns to step S11.

As described above, in the system according to the first embodiment, the alcohol in the exhaled breath of the user who has returned home is detected by the sensor, and the estimation is made as to whether or not the user drinks the alcohol after returning home based on the result of the detection. In addition, the stock amount of the alcoholic beverage is updated based on the result of the estimation, and additional orders is placed autonomously. With such a configuration, the user is not needed to manage the stock of the alcoholic beverage by himself or herself and decide ordering timing, so that usability can be improved.

In the first embodiment, in a case where the stock amount of the alcoholic beverage is less than the predetermined value, an order for the alcoholic beverage is automatically placed, but instead of placing an order for the alcoholic beverage, a recommendation may be made to the user. In this case, placing an order for the alcoholic beverage may be executed after waiting for the user's response.

### Second Embodiment

In the first embodiment, the consumption of the alcoholic beverage is estimated based on whether or not the alcohol is detected in the exhaled breath of the user.

On the other hand, the amount of alcohol drinking after returning home may be affected by the amount of alcohol drinking before returning home. For example, on days when the user drinks the alcohol a lot outside, the user does not drink the alcohol at home, and on the contrary, when the user drinks the alcohol a little outside, the user may drink the alcohol again after returning home.

The second embodiment corresponds to these cases, in which the consumption of the alcoholic beverage that is expected to be consumed by the user after returning home is estimated based on the concentration of the alcohol detected when the user has returned home.

The second embodiment is different from the first embodiment in that data in which the detected concentration of the alcohol is associated with the estimated consumption is stored as the estimation data and used in the processing of step S14.

Further, in the second embodiment, the controller 101 acquires the sensor data in the period of time when the user is expected to return home, and performs collation with the estimation data by using the value indicating the highest concentration of the alcohol acquired in the period of time. For example, in the case of the example of FIG. 5B, the value acquired at the timing indicated by reference numeral 501 is used to perform collation with the estimation data.

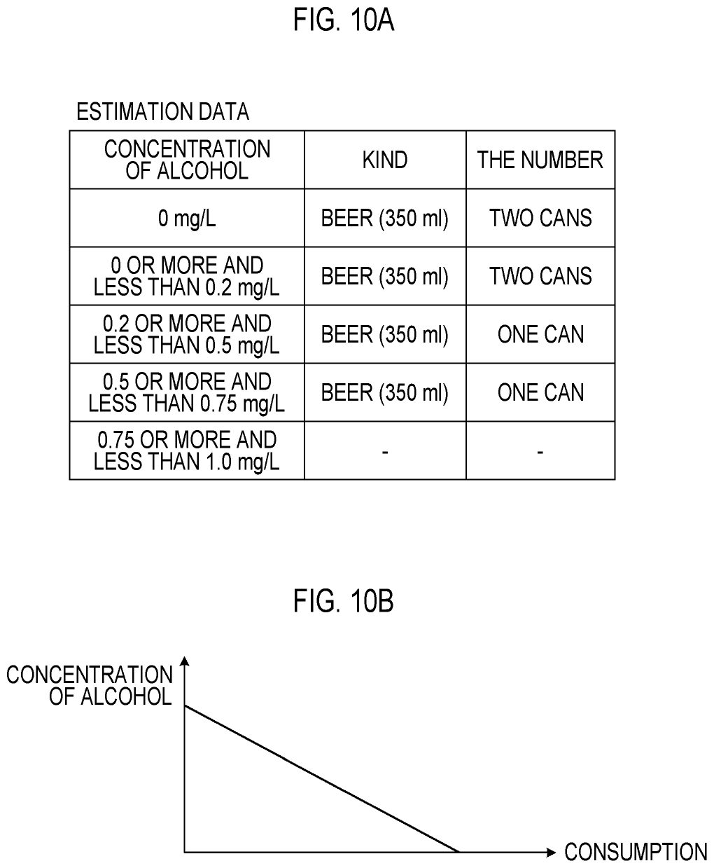

FIG. 10A shows the example of the estimation data in the second embodiment. In the case of this example, for example, in a case where the maximum value of the detected concentration of the alcohol is 0.1 mg/L, the estimation is made that the user consumes two cans of beer after returning home.

A discrete value is used as the concentration of the alcohol in the example of FIG. 10A, but the estimation data may be data in which the correlation between the detected concentration of the alcohol and the consumption of the alcoholic beverage is represented by continuous values, as shown in FIG. 10B.

Further, in the example of FIG. 10A, the alcoholic beverage is one kind, but the user may consume the kinds of the alcoholic beverages.

It is preferable that such estimation data be generated based on the drinking record of the user at home. For example, the estimation data can be generated by acquiring the concentration of the alcohol acquired when the user has returned home and the drinking record after returning home (for example, the kind of the alcoholic beverage and the amount of alcohol drinking). The drinking record may be input by the user or acquired by sensing. Further, the generated estimation data may be continuously updated by continuously acquiring the record (for example, for several days) and feeding back.

Also, the estimation data may be a machine learning model. For example, by executing machine learning with the concentration of the alcohol in the exhaled breath as input data and the consumption of the alcoholic beverage during the day as teacher data, the machine learning model for estimating the consumption of the alcoholic beverage based on the concentration data can be obtained. In this way, by using the machine learning model obtained by learning the habit of drinking alcohol of each user, the consumption of the alcoholic beverage after returning home can be accurately estimated.

According to the second embodiment, the consumption of the alcoholic beverage after the user has returned home can be more accurately estimated.

### Modification Example

The above embodiments are merely examples, and the present disclosure can be implemented with appropriate modifications within a range not departing from the gist of the present disclosure.

For example, the processing and means described in the present disclosure can be freely combined and implemented as long as no technical inconsistency occurs.

Further, in the description of the embodiments, the example in which the estimation data is generated based on the past drinking record is described, but the estimation data may be generated by using data other than the past record. For example, the blood alcohol concentration of the user may be estimated based on the concentration of the alcohol in the air, and the consumption of the alcoholic beverage may be estimated on the premise that “drinking the alcohol until the blood alcohol concentration reaches the predetermined value”.

Further, in the description of the embodiment, the sensor that detects the alcohol in the air is used as the sensor 200, but a sensor (smell sensor) that can detect a plurality of kinds of molecules that cause smell may be used. In this case, it is preferable that a unit or logic for extracting the smell level corresponding to the alcohol be used together.

The processing performed by one device in the description may be allocated and executed by a plurality of devices. Alternatively, the processing performed by different devices in the description may be executed by one device. In the computer system, the hardware configuration (server configuration) that realizes each function can be flexibly changed.

The present disclosure can also be realized by supplying a computer program that implements the functions described in the above embodiments to a computer, and reading and executing the program by one or more processors included in the computer. Such a computer program may be provided to the computer by a non-transitory computer-readable storage medium that can be connected to a system bus of the computer, or may be provided to the computer via a network. The non-transitory computer-readable storage medium includes, for example, any type of disk, such as a magnetic disk (floppy (registered trademark) disk or hard disk drive (HDD)), an optical disk (CD-ROM, DVD disk, or Blu-ray disk), read only memory (ROM), random access memory (RAM), EPROM, EEPROM, magnetic card, flash memory, optical card, and any type of media suitable for storing electronic instructions.

What is claimed is:

1. An information processing device comprising a controller configured to execute
detecting an alcohol contained in exhaled breath of a user who has returned home by using an alcohol sensor, and
estimating consumption of an alcoholic beverage by the user after returning home based on a result of the detection of the alcohol.

2. The information processing device according to claim 1, wherein the controller is configured to perform the detection by using the alcohol sensor that is installed at an entrance of home of the user.

3. The information processing device according to claim 1, wherein the controller is configured to, in a case where the alcohol is not detected from the exhaled breath of the user, estimate that a predetermined amount of the alcoholic beverage is consumed by the user after returning home.

4. The information processing device according to claim 1, wherein the controller is configured to estimate that an amount of the alcoholic beverage is consumed by the user after returning home is different between a case where the alcohol is detected from the exhaled breath of the user and a case where the alcohol is not detected from the exhaled breath of the user.

5. The information processing device according to claim 1, wherein the controller is configured to acquire a concentration of the alcohol contained in the exhaled breath of the user by using the alcohol sensor.

6. The information processing device according to claim 5, wherein the controller is configured to, in a case where the alcohol is detected from the exhaled breath of the user, estimate that an amount of the alcoholic beverage corresponding to the acquired concentration of the alcohol is consumed by the user after returning home.

7. The information processing device according to claim 6, further comprising a storage unit configured to store a model for estimating the consumption of the alcoholic beverage by the user after returning home based on the acquired concentration of the alcohol.

8. The information processing device according to claim 7, wherein the controller is configured to further acquire an actual value of the consumption of the alcoholic beverage consumed by the user after returning home.

9. The information processing device according to claim 8, wherein the model is updated based on the concentration of the alcohol acquired when the user has returned home and the actual value.

10. The information processing device according to claim 1, wherein the controller is configured to perform the detection in a predetermined period of time when the user is expected to return home, and perform the estimation based on a result of the detection in the predetermined period of time.

11. An information processing system comprising:
an alcohol sensor installed at home of a user and configured to detect an alcohol contained in exhaled breath of the user; and
a controller,
wherein the controller is configured to execute
detecting the alcohol contained in the exhaled breath of the user who has returned home by using the alcohol sensor, and
estimating consumption of an alcoholic beverage by the user after returning home based on a result of the detection of the alcohol.

12. The information processing system according to claim 11, wherein the alcohol sensor is a sensor installed at an entrance of home of the user.

13. The information processing system according to claim 11, wherein the controller is configured to, in a case where the alcohol is not detected from the exhaled breath of the user, estimate that a predetermined amount of the alcoholic beverage is consumed by the user after returning home.

14. The information processing system according to claim 11, wherein the controller is configured to estimate that an amount of the alcoholic beverage is consumed by the user after returning home is different between a case where the alcohol is detected from the exhaled breath of the user and a case where the alcohol is not detected from the exhaled breath of the user.

15. The information processing system according to claim 11, wherein the controller is configured to acquire a concentration of the alcohol contained in the exhaled breath of the user by using the alcohol sensor.

16. The information processing system according to claim 15, wherein the controller is configured to, in a case where the alcohol is detected from the exhaled breath of the user, estimate that an amount of the alcoholic beverage corresponding to the acquired concentration of the alcohol is consumed by the user after returning home.

17. The information processing system according to claim 16, further comprising a storage unit configured to store a model for estimating the consumption of the alcoholic beverage by the user after returning home based on the acquired concentration of the alcohol.

18. The information processing system according to claim 17, wherein the controller is configured to further acquire an actual value of the consumption of the alcoholic beverage consumed by the user after returning home.

19. The information processing system according to claim 18, wherein the model is updated based on the concentration of the alcohol acquired when the user has returned home and the actual value.

20. An information processing method comprising:
a step of sensing a user who has returned home;
a step of detecting an alcohol contained in exhaled breath of the user who has returned home; and
a step of estimating consumption of an alcoholic beverage by the user after returning home based on a result of the detection of the alcohol.

21. A program that causes a computer to execute the information processing method according to claim 20.
